(19) United States
(12) Patent Application Publication
(10) Pub. No.: US 2007/0282155 A1
(43) Pub. Date: Dec. 6, 2007

(54) Seed-Holding Device

(76) Inventors: Rohit Mehta, Clifton, VA (US); Markus Muehlheim, New Port Richey, FL (US)

Correspondence Address: BEST MEDICAL INTERNATIONAL, INC.;ATTN: RAY, 7643 FULLERTON ROAD, SPRINGFIELD, VA, 22153, US

(21) Appl. No.: 11/422,469

(22) Filed: Jun. 6, 2006

(51) Int. Cl. A61M 36/00; A61N 5/00

(52) U.S. Cl. 600/7

(57) ABSTRACT

A seed-holding device comprises of a transfer device having a main body and a pusher. The device also includes a holder for engaging the transfer device; the transfer device has a closed proximal end, an open distal end, an expansive spring, and a pusher connecting means. The expansive spring is located between the closed proximal end and the pusher connecting means. The seed-holding device, when loaded, has no parts that can be easily broken by extending beyond the proximal end of the transfer device.

The present invention is directed to a seed-holding device. More particularly, the invention relates to a seed-holding device that overcomes problems in existing seed-holding devices

### BACKGROUND OF THE INVENTION

Seed-holding devices for use in radiation therapy are known. Two representative devices are shown in U.S. Pat. No. 5,860,909 to Mick et al. and U.S. Pat. No. 6,682,471 to Steele, Sr. et al. The patents are owned, respectively by Mick Radio Nuclear Instruments, Inc., and Mentor Corporation, two companies in the business of making and selling seed-holding devices

A commercial device know as the Mick ® 15 (the number representing the maximum number of seeds that the device can hold) has a seed spring clamp that can be knocked off or fall apart. Moreover the pusher in the Mick ® 15 device has a tendency to break when loaded; the parts of the device have a tendency to become loose when the pusher is carried loaded. The tendency to break when loaded comes about because a shaft on the pusher extends beyond the end of the transfer device.

In a device known as the Mentor 20 (the number here too representing the maximum number of seeds that can be held by the device), the pusher is connected to an indicator that extends through a proximal end of the device. Thus, the device has a tendency to break when loaded because the indicator extends beyond the end of the device. The device, furthermore, is hard to assemble. The purpose of the present invention is to provide a seed-carrying device that does not come apart and has nothing to catch or break when in use, thus overcoming certain problems in known seed-holding devices. The invention relates to a seed-holding device having an arrangement in a transfer device therefore that overcomes the above-discussed problems of the prior art.

### BRIEF DISCUSSIONS OF THE DRAWINGS

FIG. 1 is a perspective view of a seed-holding device of the present invention.

FIG. 2 is a cutaway view of a seed-holding device of the present invention.

FIG. 3(a) is an axial cross-sectional view of a seed-holding device of the present invention.

FIG. 3(b) is a side view of a seed-holding device of the present invention,

FIG. 3(C) is a front-end view of a seed-holding device of the present invention.

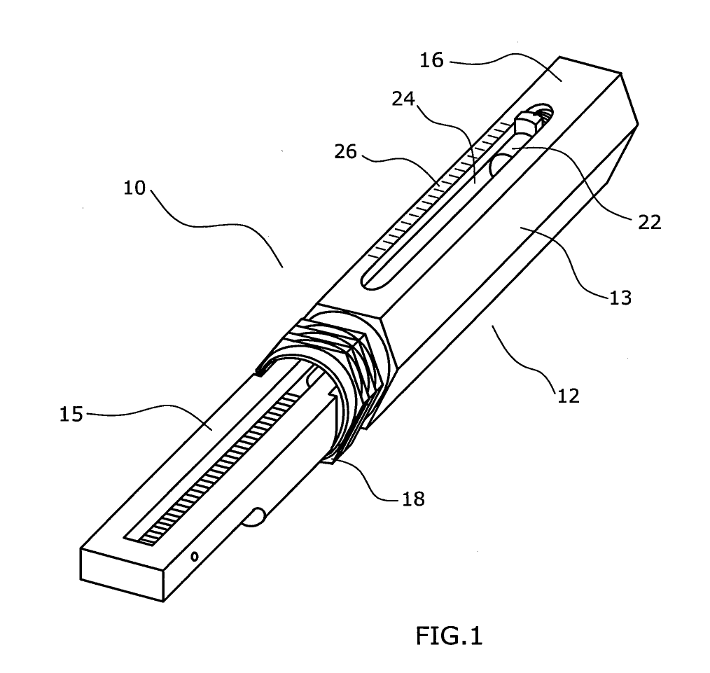

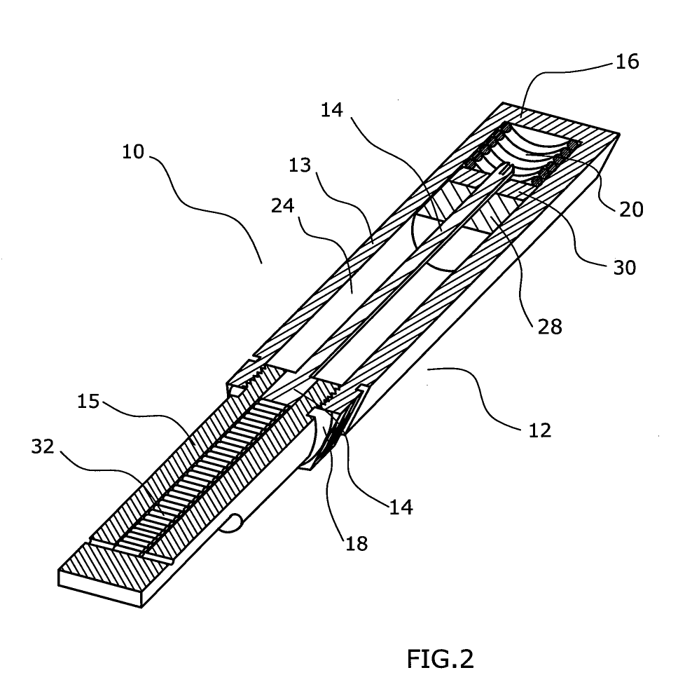

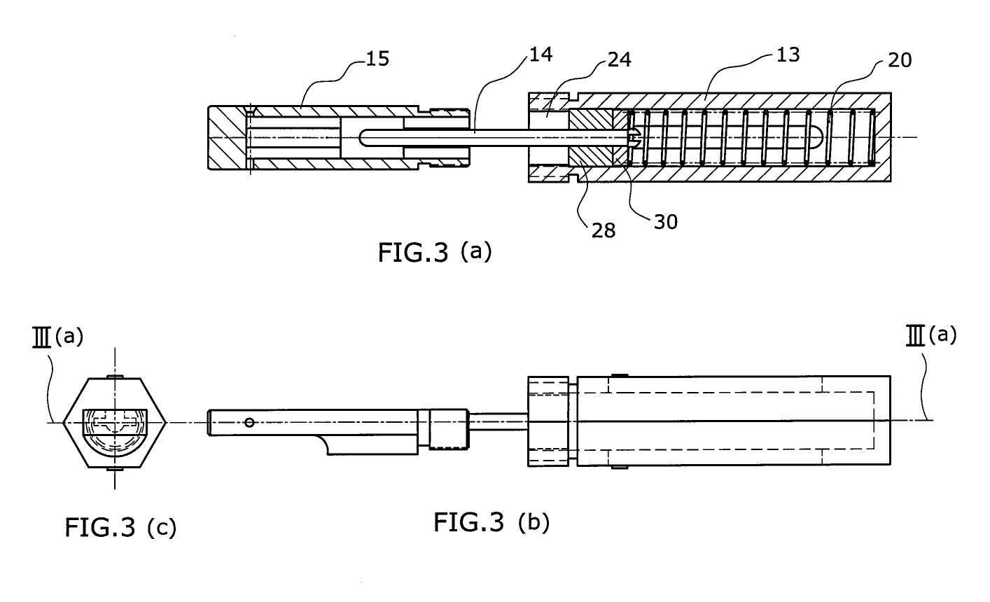

### DETAILED DISCUSSION OF THE INVENTION

The seed-holding device 10 has a transfer device 12 configured to receive at least one seed 32, the transfer device 12 having a main body 13 and a pusher 14 that slides in the main body 13. The main body has a cross-section that may be round, hexagonal, octagonal, pentagonal, or of any other desired shape.

The holder 15 is configured to engage the transfer device 12. The transfer device 12 has a closed proximal end 16 and an open distal end 18. Expansive spring 20 and pusher connecting means 22 are present in the transfer device 12. The expansive spring 20 is located between the closed proximal end 16 and the pusher connecting means 22.

The pusher connecting means 22 travels in an opening 24 in a longitudinal surface of the transfer device 12 which can contain gradations 26 along the opening 24 to indicate the number of seeds that are present in the seed-holding device.

The pusher connecting means 22 has an arm 28 in association with the opening 24 in the longitudinal surface of the transfer device 12 and also has a plug 30 located on a surface facing the closed proximal end of the transfer device 12.

As one readily can see, the seed-holding device of the present invention, when loaded, has no parts that extend beyond the major parts of the device and thus do not lend themselves to breaking.

What is claimed is:

1. A seed-holding device comprising:
a. a transfer device configured to receive at least one seed, comprising a main body and a pusher that slides in the main body, and
b. a holder configured to engage the transfer device, the transfer device having a closed proximal end and the pusher connecting means.

2. The seed-holding device of claim 1, wherein the pusher connecting means travels in an opening in a longitudinal surface of the transfer device.

3. The seed-holding device of claim 2 further comprising gradations along the opening in the longitudinal surface of the transfer device.

4. The seed-holding device of claim 2, wherein the pusher connecting mean has an arm in association with the opening in the longitudinal surface of the transfer device.

5. The seed-holding device of claim 2, wherein the pusher connecting means has a plug located on a surface facing the closed proximal end of the transfer device.
